(19) United States
(12) Patent Application Publication
(10) Pub. No.: US 2025/0152950 A1
(43) Pub. Date: May 15, 2025

(54) IMPLANTABLE MEDICAL DEVICES AND HEADER ASSEMBLIES FOR USE WITH SAME

(71) Applicant: The Alfred E. Mann Foundation for Scientific Research, Valencia, CA (US)

(72) Inventors: Joseph L. Calderon, Santa Clarita, CA (US); Jaime Gutierres, Santa Clarita, CA (US); Edward Hillery, Valencia, CA (US)

(73) Assignee: The Alfred E. Mann Foundation for Scientific Research, Valencia, CA (US)

(21) Appl. No.: 18/601,594

(22) Filed: Mar. 11, 2024

Related U.S. Application Data
(60) Provisional application No. 63/599,487, filed on Nov. 15, 2023.

(51) Int. Cl. A61N 1/375; A61N 1/05

(52) U.S. Cl. CPC A61N 1/3758; A61N 1/0556; A61N 1/3754

(57) ABSTRACT

An implantable medical device including an electronics housing, circuitry within the electronics housing, and a header assembly secured to the electronics housing. The header assembly includes a hollow header body defining an internal volume, a header assembly connector located within the internal volume, operably connected to the circuitry, defining an interior configured to receive a lead connector and an exterior, a hard fill within the internal volume, and a seal between the exterior of the header assembly connector and the hard fill.

### CROSS-REFERENCE TO RELATED APPLICATION

This application claims the benefit of U.S. Provisional Application No. 63/599,487, filed Nov. 15, 2023, and entitled “IPG Connector Headers for Implantable Medical Devices,” which is incorporated herein by reference.

### BACKGROUND OF THE INVENTIONS

### 1. Field of Inventions

The present inventions relate generally to implantable medical devices such as, for example, implantable tissue stimulators.

### 2. Description of the Related Art

Implantable medical devices such as, for example, implantable tissue stimulators, implantable drug delivery devices and implantable physiological monitors, frequently include an implantable electronics component and a lead component. The implantable electronics component may include a hermetically sealed electronics housing and a header assembly with an electrical connector that is mounted on the electronics housing, while the lead component may have an electrical connector that is configured to mate with the electronics component electrical connector. The electronics component electrical connector may be connected to electronics within the electronics housing by way of feedthrough leads (e.g., pins or wires) that extend through the housing.

Although the present inventions are not so limited, the inventions are described herein in the exemplary context of implantable tissue stimulators, which may include an implantable pulse generator (“IPG”) and an electrode lead. Implantable tissue stimulators are used to treat a wide variety of medical conditions. One such condition is obstructive sleep apnea (OSA), which is a highly prevalent sleep disorder that is caused by the collapse of or increase in the resistance of the pharyngeal airway, often resulting from tongue obstruction. Here, nerve fascicles of the hypoglossal nerve (HGN) that innervate the intrinsic and extrinsic muscles of the tongue are stimulated in a manner that prevents retraction of the tongue, which would otherwise close the upper airway during the inspiration portion of the respiratory cycle. Other exemplary medical conditions that may be treated with tissue stimulations include, but are not limited to, chronic pain syndrome, which may be treated spinal cord stimulation, neurological disorders, which may be treated with deep brain stimulation, and slow or irregular heartbeats, which may be treated with a pacemaker.

### SUMMARY

The present inventors have determined that implantable medical devices are susceptible to improvement. For example, some conventional electronics component header assemblies include header body that defines an interior volume and a header assembly electrical connector (or “HA connector”), with an alternating series of conductive members and non-conductive members, that is located within the interior volume. In some instances, the portion of the interior volume that is not occupied by the HA connector and associated structures is filled with liquid epoxy that, upon hardening, prevents movement of the HA connector. The present inventors have determined that preventing movement of the HA connector in this manner is less than optimal because the liquid epoxy frequently passes through tiny gaps between the conductive members and non-conductive members. The epoxy contaminates the interior of the HA connector and renders the associated header assembly unusable.

An implantable medical device in accordance with at least one of the present inventions includes an electronics housing, circuitry within the electronics housing, and a header assembly secured to the electronics housing. The header assembly includes a hollow header body defining an internal volume, a header assembly connector located within the internal volume, operably connected to the circuitry, defining an interior configured to receive a lead connector and an exterior, a hard fill within the internal volume, and a seal between the exterior of the header assembly connector and the hard fill.

A method in accordance with at least one of the present inventions includes the steps of sealing the exterior of a header assembly connector that includes a plurality of conductive members and a plurality of non-conductive members, positioning the header assembly connector with the sealed exterior within an interior volume of an implantable medical device header body, and after the positioning step, transferring liquid state hard fill material into the interior volume of the implantable medical device header body.

There are a number of advantages associated with such apparatus and methods. By way of example, but not limitation, the seal on the header assembly connector prevents liquid state hard fill material from contaminating the interior of the header assembly connector and rendering the associated header assembly unusable.

### BRIEF DESCRIPTION OF THE DRAWINGS

Detailed descriptions of exemplary embodiments will be made with reference to the accompanying drawings.

FIG. 1 is a plan view of a system including a tissue stimulator and related components in accordance with one embodiment of a present invention.

FIG. 2 is a block diagram of the system illustrated in FIG. 1.

FIG. 3 is a plan view showing the nerve cuff illustrated in FIG. 1 on an HGN GM branch.

FIG. 4 is a side view of a portion of the tissue stimulator illustrated in FIG. 1.

FIG. 5 is a side, partial section and cutaway view of a portion of the tissue stimulator illustrated in FIG. 1.

FIG. 6 is a top view of a portion of the tissue stimulator illustrated in FIG. 1.

FIG. 7 is a side view of a portion of the tissue stimulator illustrated in FIG. 1.

FIG. 8 is a section view of a portion of the tissue stimulator illustrated in FIG. 1 in a disconnected state.

FIG. 9 is a section view of a portion of the tissue stimulator illustrated in FIG. 1 in a connected state.

FIG. 10 is an exploded side view showing the assembly of a portion of the tissue stimulator illustrated in FIG. 1.

FIG. 11 is a side view of a portion of the tissue stimulator illustrated in FIG. 1.

FIG. 12 is a side view of a portion of the tissue stimulator illustrated in FIG. 1.

FIG. 13 is a side, partial section view of a portion of the tissue stimulator illustrated in FIG. 1.

FIG. 14 is a side, partial section view of a portion of the tissue stimulator illustrated in FIG. 1.

FIG. 15 is a side, partial section view of a portion of the tissue stimulator illustrated in FIG. 1.

FIG. 16 is a side, partial section view of a portion of the tissue stimulator illustrated in FIG. 1.

FIG. 17 is a flow chart showing a method in accordance with one embodiment of a present invention.

FIG. 18 is a side view of a portion of a tissue stimulator in accordance with one embodiment of a present invention.

### DETAILED DESCRIPTION OF THE EXEMPLARY EMBODIMENTS

The following is a detailed description of the best presently known modes of carrying out the inventions. This description is not to be taken in a limiting sense, but is made merely for the purpose of illustrating the general principles of the inventions. For example, although described in the exemplary context of tissue stimulators associated with sleep apnea treatment, the present inventions are not so limited and are applicable to implantable tissue stimulators that are configured to treat other medical conditions, as well as other implantable medical devices such as implantable drug delivery devices and implantable physiological monitors.

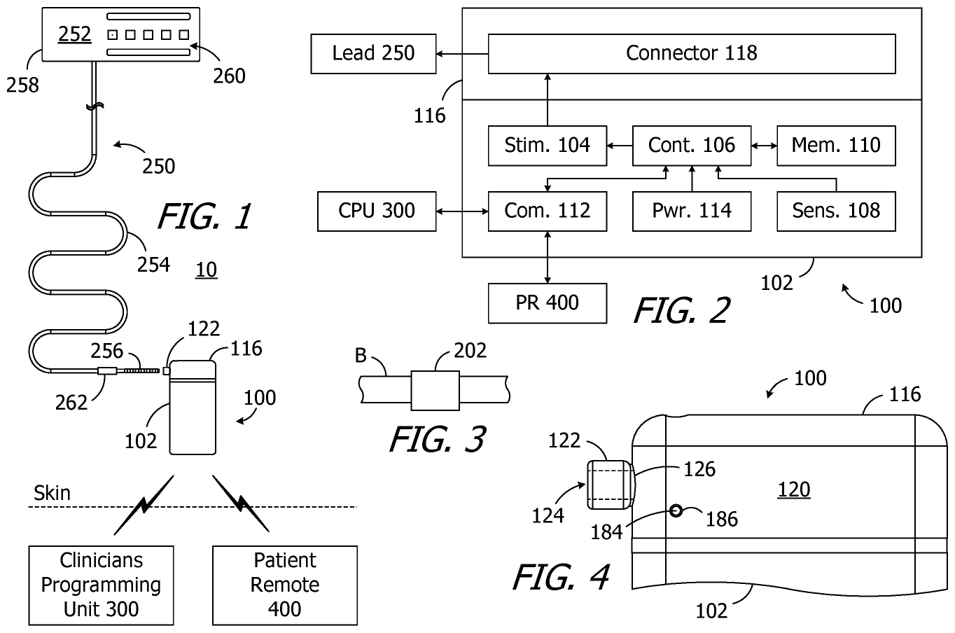

Referring to FIGS. 1-4, an implantable tissue stimulator 10 in accordance with one embodiment of a present invention includes an implantable pulse generator (“IPG”) 100 and an electrode lead 250. The implantable tissue stimulator 10 may be incorporated into a system that also includes a clinician's programming unit 300, a patient remote 400 and/or an IPG charger (not shown) in some instances.

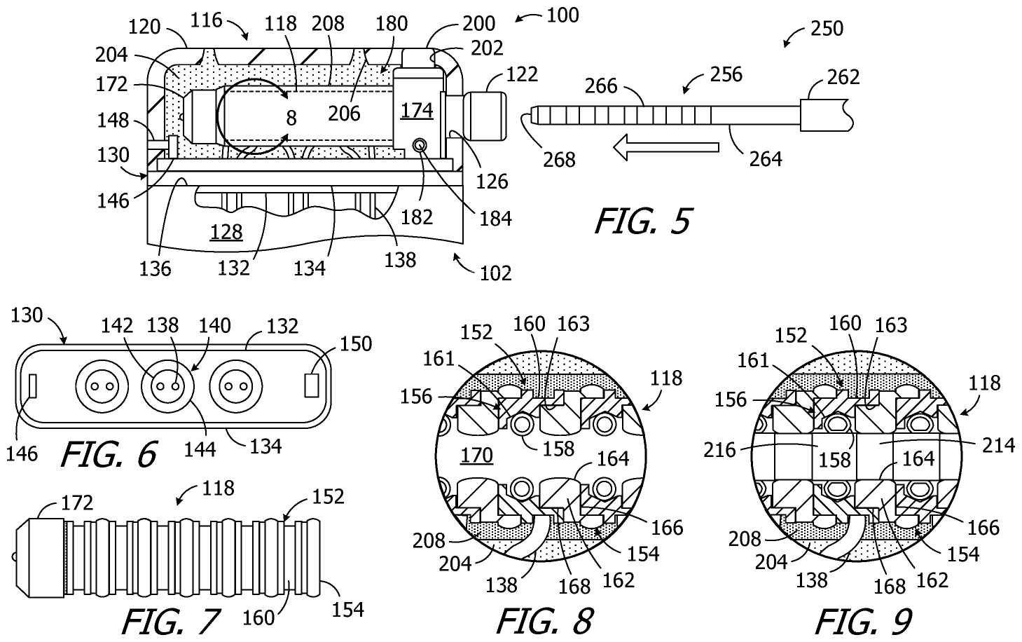

The exemplary IPG 100 includes a hermetically sealed electronics housing 102 in which various circuitry (e.g., stimulation circuitry 104, control circuitry 106, sensing circuitry 108, memory 110 and communication circuitry 112) and a power supply 114 is located. The exemplary IPG 100 also includes a header assembly 116 that is secured to the electronics housing 102 and that may be composed of a header assembly connector 118 (or “HA connector” or “connector” or “electrical connector”), a header body 120 that mounts the HA connector to the exterior of the electronics housing, and a strain relief 122 that defines a lead connector port 124 and extends through an opening 126 in the header body 120. The HA connector 118 is connected to the stimulation circuitry 104 by way of feedthrough leads 138 (FIGS. 5 and 6). The header body 120 is hollow and may be formed from a polymer such as cast epoxy or molded plastic. As discussed in greater detail below, the header body internal volume includes a hard fill that prevents movement of the HA connector 118 within the header body 120 and a seal is formed over the exterior of the HA connector to prevent the hard fill material from contaminating the interior of the HA connector. As used herein, a “hard fill” is a material that is transferred into the unoccupied portion of a volume in a liquid (or otherwise flowable) state and that, once it has hardened, will occupy all (or at least substantially all) of the previously unoccupied portion of the volume, and prevent movement of adjacent structures within the volume.

The exemplary electrode lead 250 illustrated in FIG. 1 includes a nerve cuff 252 and a lead body 254 that couples the nerve cuff 252 to the IPG 100 by way of a lead electrical connector (or “lead connector”) 256 on the proximal end of the lead body 254. The exemplary nerve cuff 252 is configured in such a manner that it may be circumferentially disposed around, and provide stimulation energy to, either the HGN trunk or a HGN branch (e.g., the HGN GM branch B shown in FIG. 3). The nerve cuff 252, which is shown in a unfurled state in FIG. 2 and is pre-set to a furled state, includes a cuff body 258 and a plurality of electrically conductive contacts (or “contacts”) 260. A strain relief 262 is located adjacent to the lead connector 256. The lead body 254 in the illustrated implementation includes a plurality of S-shaped sections in order to provide strain relief and accommodate body movement at the location within the neck where the lead body 254 is implanted, thereby reducing the likelihood that damage to HGN, as well as fatigue damage of the lead body 254, that may result from neck movements. The lead body may also be straight in other implementations. The cuff body 258 in the exemplary implementation has a stimulation region with the contacts 260 and a compression region with no contacts. The compression region wraps around at least a portion of the stimulation region when the nerve cuff 102 is in the pre-shaped furled state, as well as in slightly larger, expanded and less tightly furled states, thereby improving the electrical connection between the contacts 260 and the nerve. Suitable cuff body materials may be biologically compatible, electrically insulative and elastic and include, but are not limited to silicone, polyurethane and styrene-isobutylene-styrene (SIBS) elastomers. The contacts 260 may differ in size and shape (as shown) or may be the same size and/or shape. Suitable contact materials includes, but are not limited to, platinum-iridium and palladium. The present electrode leads may include other types of nerve cuffs, such as helical and axial nerve cuffs, and various specific examples of nerve cuffs are illustrated and described in U.S. Pat. Pub. Nos. 2018/0318577A1, 2018/0318578A1, 2019/0060646A1, 2019/0282805A1, 2522/0313987A1 and 2523/10510A1, which are incorporated herein by reference in their entirety. The electrode leads of the present tissue stimulators may also include nerve paddles and nerve strips in place of the nerve cuffs.

In the exemplary context of OSA treatment, the circuitry within the electronics housing 102 housing may be configured to, for example, deliver stimulation energy to the HGN by way of the nerve cuff 252. Here, the control circuitry 106 may apply stimulation energy to either the HGN trunk or an HGN branch (e.g. the HGN GM branch) in various stimulation methodologies by way of the cuff 252 when the patient is in the inspiratory phase of respiration, and other conditions for stimulation are met, thereby causing anterior displacement of the tongue to keep the upper airway unobstructed. The control circuitry 106 causes the stimulation circuitry 104 to apply stimulation in the form of a train of stimulation pulses during these inspiratory phases of the respiratory cycle (or slightly before the inspiration and ending at the end of inspiration) and not the remainder of the respiration cycle. The train of stimulus pulses may be set to a constant time duration or may change dynamically based on a predictive algorithm that determines the duration of the inspiratory phase of the respiratory cycle.

The sensing circuitry 108 may be connected to one or more sensors (not shown) that are contained within the housing 102. Alternatively, or in addition, the sensors may be affixed to the exterior of the housing 102 or positioned at a remote site within the body and coupled to the IPG 100 with a connecting lead. The sensing circuitry 108 can detect physiological artifacts that are caused by respiration (e.g., motion or ribcage movement), which are proxies for respiratory phases, such as inspiration and expiration or, if no movement occurs, to indicate when breathing stops. Suitable sensors include, but are not limited to, inertial sensors, bioimpedance sensors, pressure sensors, gyroscopes, ECG electrodes, temperature sensors, GPS sensors, and combinations thereof. The memory 110 stores data gathered by the sensing circuitry 108, programming instructions and stimulation parameters. The control circuitry 106 analyzes the sensed data to determine when stimulation should be delivered. The communication circuitry 112 is configured to wirelessly communicates with the clinician's programming unit 300 and patient remote 400 using radio frequency signals.

It should also be noted that although the exemplary tissue stimulator 10 illustrated in FIGS. 1-4 includes a single lead, the present inventions are not so limited. Other embodiments may, for example, include a pair of electrode leads 250 for bilateral HGN stimulation and an IPG (not shown) with two HA connectors 118.

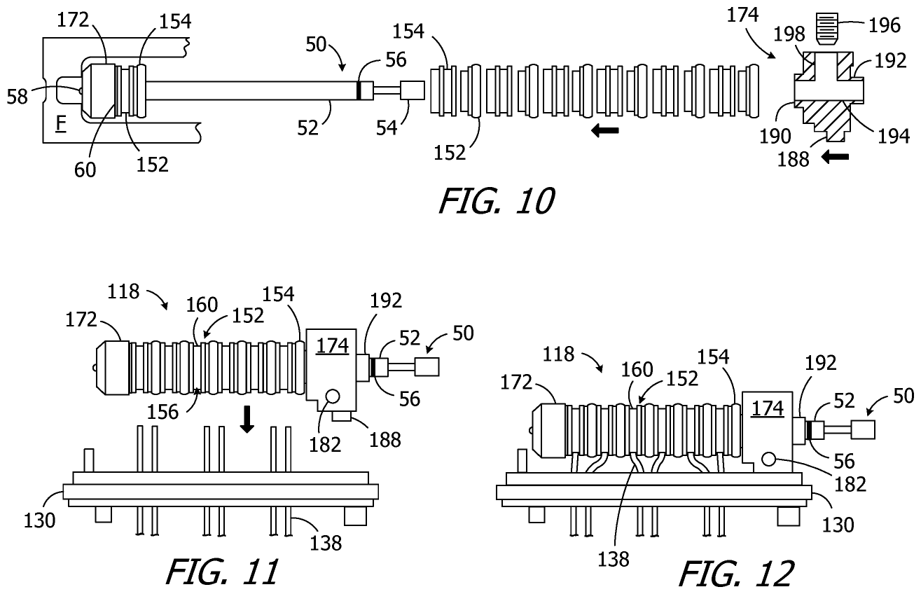

Turning to FIGS. 5 and 6, the exemplary hermetically sealed electronics housing 102 includes an electronics container 128 as well as a cover 130 with a central portion 132 and a flange 134 that extends outwardly from the central portion. The flange 134 rests on and is welded or otherwise bonded to the container rim 136 to form the sealed housing 102. Suitable materials for the container 122 and cover 130 include, but are not limited to, an electrically conductive, biocompatible material such as titanium. The stimulation circuitry 104 within the electronics housing 102 may be connected to components outside the housing by way of feedthrough leads (e.g., pins or wires) 138 that extend through the cover central portion 126, or by other suitable instrumentalities. In the illustrated embodiment, the feedthrough leads 138 are part of feedthrough assemblies 140 that each include a pair of feedthrough leads 138, a ceramic insulator 142 through which the feedthrough leads extend, and a base member 144 in which the ceramic insulator is mounted. The feedthrough assemblies 140 extend through apertures in the cover central portion 132 and are welded or otherwise bonded thereto. The exemplary cover 130 also includes an anchor post 146 with an aperture (not shown) for a pin 148 that is used to secure the header body 120 to the cover and a recess 150 for the connector block post 188 (discussed below with reference to FIG. 10).

As illustrated in FIGS. 7-9, the exemplary HA connector 118 includes an alternating series of electrically conductive members (“conductive members”) 152 and electrically non-conductive members (“non-conductive members”) 154. The electrically conductive and non-conductive members 152 and 154 in the illustrated implementation are respectively configured such that adjacent conductive and non-conductive members may be (and are) secured to one another by way of an interference fit. The exemplary conductive members 152 include a generally annular main body 156 and an annular coil spring 158. The annular main body 156 has a lead recess 160 to which a lead 138 is welded or otherwise connected, a spring recess 161 for the coil spring 158, and a recess 163 for a portion of the non-conductive member 154. The non-conductive members 154 include a generally annular main body 162, which defines an engagement surface 164, and recesses 166 and 168 for the conductive members 152. The conductive members 152 and non-conductive members 154 together define an internal lumen/receptacle 170 for the lead connector 256 which, as illustrated in FIG. 5, has an electrically non-conductive member 264 and a plurality of spaced electrically conductive contacts 266 on the non-conductive member. The HA connector 118 also includes an end cap 172 that is contacted by the lead connector free end 268 when the when the lead connector 256 is fully inserted into the HA connector receptacle 170. The respective dimensions of the components of the HA connector 118 and the lead connector 256 are such that, when the lead connector 256 is fully inserted into the HA connector receptacle 170, the contacts 266 will be aligned with the conductive members 152 and will compress the coil springs 158 while the portions of the non-conductive member 264 will be aligned with the non-conductive member engagement surfaces 164 and will compress the main bodies 162.

The present inventions are not limited to any particular HA connectors, conductive members, non-conductive members and/or materials. By way of example, but not limitation, suitable materials for the conductive member main body 156 include MP35N® steel, suitable materials for the coil spring 158 include platinum-iridium, and suitable materials for the non-conductive members 154 include silicone rubber.

Referring again to FIG. 5, the HA connector 118 and the strain relief 122 are mounted on a connector block 174 in the illustrated implementation. The connector block 174 is located, together with the HA connector 118, within a portion of the internal volume 180 of the hollow header body 120 and is secured to the header body 120 and to the cover 130. With respect to connection to the cover 130, the connector block 174 includes apertures 182 for pins 184 that extend through corresponding apertures 186 (FIG. 4) on opposite sides of the header body 120. Turning to FIG. 10, the connector block 174 also includes a post 188 than is inserted in the cover recess 150 (FIG. 6), and the connector block may be welded to the cover 130 after the post is inserted into the recess. The connector block 174 also includes a mounting post 190 for the HA connector 118, a mounting post 192 for the strain relief 122, a lumen 194 for the lead connector 256 (and an assembly pin 50 that is used during assembly) that extends through the connector block, a set screw 196, and threaded set screw lumen 198 that extends to the lumen 194. Access to the set screw 196 is provided by way of a septum 200 (FIG. 5) that extends into an opening 202 in the header body 120.

As is illustrated for example in FIGS. 5, 8 and 9, the remainder of the internal volume 180 is occupied by a hard fill 204 that is initially in a liquid state and that hardens after being injected into the internal volume by way of an aperture 206, thereby preventing movement of the HA connector 118 within the header body 120. A seal 208 is formed over the exterior of the HA connector 118, i.e., is formed completely over and around the conductive members 152 and non-conductive members 154, prior to the placement of the header body 120 onto the cover 130. The seal material is allowed to harden into the seal 208 prior to the transfer of liquid-state hard fill material into the internal volume 180, which prevents the liquid-state hard fill material from passing through any gaps between the conductive members 152 and non-conductive members 154 and contaminating the interior of the HA connector 118.

The present inventions are not limited to any particular hard fill and seal materials. Suitable materials for the hard fill 204 include, but are not limited to, biocompatible epoxies such as EPO-TEK® 301-2 two-part epoxy. Suitable materials for the seal 208 include materials that are more viscous than the hard fill materials and are sufficiently viscous to prevent ingress into the HA connector 118 when applied. Specific examples include, but are not limited to, silicone adhesives such as NuSil® MED-2000. It should also be noted that, once cured, the seal materials may be softer and lower modulus than the hard fill materials because they are not used to provide structural stability, which is the function of the hard fill materials.

One exemplary method of assembling the present header assembly 116 is illustrated in FIGS. 10-16. Referring first to FIG. 10, the HA connector 118 may in some instances be assembled through use of a fixture F (partially shown) and the exemplary assembly pin 50, which has a main body 52, a depth marker 54, a handle 56 and a tip 58. The assembly pin 50 is inserted into the end cap 172 and temporarily secured thereto with a small amount of adhesive (not shown). The first conductive member 152 is then placed onto the assembly pin main body 52 and secured to the end cap 172 with adhesive 60. Next, the first non-conductive member 154 may be placed onto the assembly pin main body 52 and pressed against the first conductive member 152 until the non-conductive member main body 162 is within the conductive member recess 163 and abuts the conductive member main body 156 (FIG. 8). The next conductive member 152 may then be pressed against the first non-conductive member 154 until the conductive member main body 156 is within the recesses 166 and abuts the non-conductive member main body 162 (FIG. 8). This alternating process will continue until all of the conductive and non-conductive members 152 and 154 are pressed against one another on the pin 50.

Next, the connector block 174 is placed on the assembly pin 50, with the assembly pin main body 52 extending though the connector block lumen 194, and pressed against the last non-conductive member 154 until the mounting post 190 is located within the recess 166 and the marker is located just outside the mounting post 192. Such positioning of the connector block 174 on the assembly pin 50 results in the conductive and non-conductive members 152 and 154 being in their intended locations within the now-completed connector 118. The position of the connector block 174 may then be fixed with the set screw 196.

Turning to FIGS. 11 and 12, the HA connector 118, connector block 174 and assembly pin 50 are positioned adjacent to the housing cover 130, with the connector block post 188 located within the cover recess 150. The connector block 174 may then be welded to the housing cover 130. The leads 138 may be positioned within and welded to the conductive member lead recesses 160.

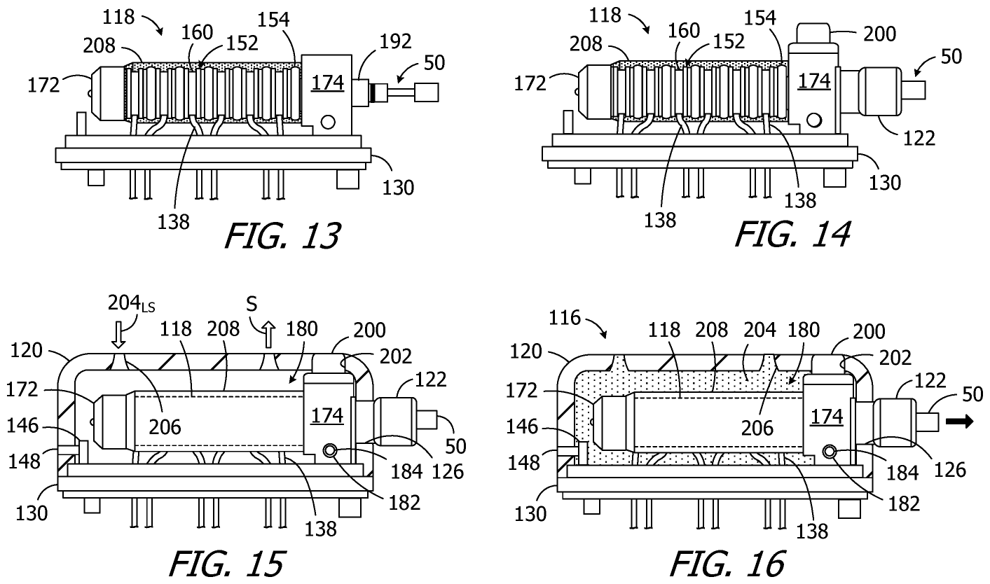

The seal 208 may then be formed over the exterior of the HA connector 118 by applying seal material (e.g., silicone adhesive) completely over and around the conductive members 152 and non-conductive members 154, and then allowing the seal material to cure into the seal 208 illustrated in FIG. 13. The septum 200 may be secured to the connector block 174 over the threaded set screw lumen 198 (FIG. 10), as shown in FIG. 14, and the strain relief 122 may be secured to the connector block mounting post 192. Silicone adhesive may be used here as well.

Referring to FIG. 15, the header body 120 may be mounted onto the housing cover 130 and secured thereto with adhesive that also creates a seal between the header body and the cover. Additionally, the pin 148 may be press-fit into the anchor post 148 to further secure the header body 120 to the cover 130. The pins 184 may be press-fit into the connector block apertures 182, by way of the apertures 186, to further secure the header body 120 to the cover 130. The liquid state hard fill material 204_{LS} may then be injected into the header body internal volume 180 by way of one of the apertures 206. Air within the internal volume 180 may also be simultaneously removed with suction S. The seal 208 formed over the exterior of the HA connector 118 will prevent the liquid state hard fill material 204_{LS} from passing through any gaps between the conductive members 152 and non-conductive members 154. The liquid state hard fill material 204_{LS} is allowed to cure (or “harden”) into a solid, and the solid-state hard fill material 204 (FIG. 16) prevents movement of the HA connector 118. The assembly pin 50 may remain secured to the connector block 174 until the fill material 204 has cured to maintain the proper position of the HA connector 118 within the header body 120 during the assembly process. The set screw 196 may then be rotated, with a tool that has been inserted through the septum 200, so that assembly pin 50 can be moved.

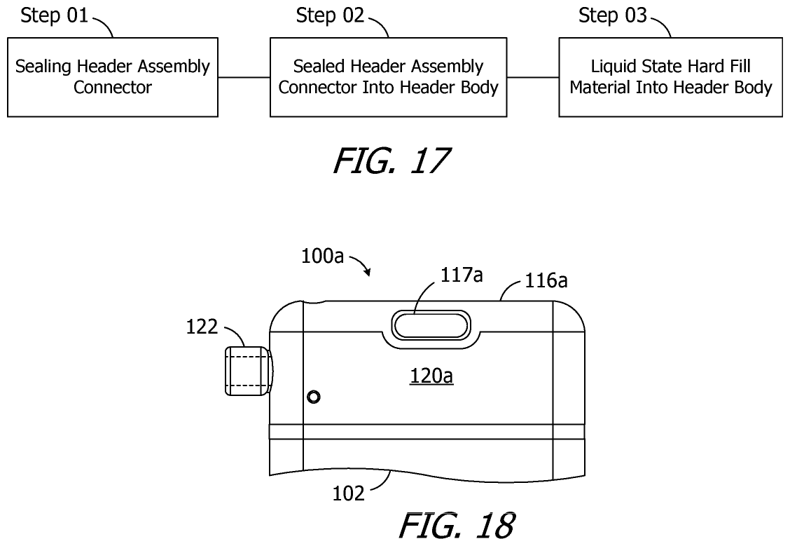

In summary, and referring to FIG. 17, the exemplary assembly method may be summarized as follows. The exterior of a header assembly connector, which includes a plurality of conductive members and a plurality of non-conductive members, is sealed (Step 01). The sealed header assembly connector is then positioned within an interior volume of an implantable medical device header body (Step 02). Liquid state hard fill material is then transferred into the interior volume of the implantable medical device header body and allowed to cure (Step 03).

Although the inventions disclosed herein have been described in terms of the preferred embodiments above, numerous modifications and/or additions to the above-described preferred embodiments would be readily apparent to one skilled in the art.

By way of example, but not limitation, the exemplary IPG 100a illustrated in FIG. 18 is essentially identical to IPG 100 and similar elements are represented by similar reference numerals. For example, the includes a header assembly 116a, with a header body 120a, that is secured to the electronics housing 102 as well as a sealed HA connector (not shown) such as that described above. Here, however, the header body 120a includes an aperture 117a for a suture or other instrumentality that may be used to anchor the IPG100a in place within the body.

It is intended that the scope of the present inventions extend to all such modifications and/or additions. The inventions include any and all combinations of the elements from the various embodiments disclosed in the specification. The scope of the present inventions is limited solely by the claims set forth below.

1. An implantable medical device, comprising:
an electronics housing;
circuitry within the electronics housing; and
a header assembly secured to the electronics housing and including
a hollow header body defining an internal volume,
a header assembly connector located within the internal volume, operably connected to the circuitry, defining an interior configured to receive a lead connector and an exterior,
a hard fill within the internal volume, and
a seal between the exterior of the header assembly connector and the hard fill.

2. The implantable medical device claimed in claim 1, wherein
the electronics housing includes a container and a cover; and
the header assembly connector is connected to the circuitry within the electronics housing by a plurality of feedthrough leads that extend through the cover.

3. The implantable medical device claimed in claim 1, wherein
the circuitry comprises stimulation circuitry.

4. The implantable medical device claimed in claim 1, wherein
the header assembly connector includes a plurality of conductive members and a plurality of non-conductive members.

5. The implantable medical device claimed in claim 1, wherein
the conductive members include an annular main body and an annular coil spring.

6. The implantable medical device claimed in claim 1, wherein
adjacent conductive members and non-conductive members are secured to one another with an interference fit.

7. The implantable medical device claimed in claim 1, wherein
the hollow header body is formed from a polymer.

8. The implantable medical device claimed in claim 1, wherein
the header assembly include a connector block located within the header body; and
the header assembly connector is mounted on the connector block.

9. The implantable medical device claimed in claim 1, wherein
the seal is formed from seal material; and
the hard fill is formed from hard fill material that is different than the seal material.

10. The implantable medical device claimed in claim 9, wherein
the seal material defines a liquid state viscosity; and
the hard fill material defines a liquid state viscosity that is less than the seal material liquid state viscosity.

11. The implantable medical device claimed in claim 9, wherein
the seal material defines a cured state modulus; and
the hard fill material defines a cured state modulus that is greater than the seal material cured state modulus.

12. The implantable medical device claimed in claim 9, wherein
the hard fill material comprises epoxy; and
the seal material comprises silicone adhesive.

13. The implantable medical device claimed in claim 1, further comprising:
an electrode lead including an elongate lead body having a proximal end and a distal end, a plurality of electrically conductive contacts associated with the distal end of the lead body, and a lead connector associated with proximal end of the lead body and having a non-conductive member and a plurality of spaced electrically conductive contacts on the non-conductive member.

14. The implantable medical device claimed in claim 13, wherein
the electrode lead includes a nerve cuff and the electrically conductive contacts are part of the nerve cuff.

15. A method, comprising the steps of:
sealing the exterior of a header assembly connector that includes a plurality of conductive members and a plurality of non-conductive members;
positioning the header assembly connector with the sealed exterior within an interior volume of an implantable medical device header body; and
after the positioning step, transferring liquid state hard fill material into the interior volume of the implantable medical device header body.

16. The method of claim 15, further comprising the step of:
securing leads to the connector prior to applying the seal.

17. The method of claim 15, wherein
sealing comprises applying seal material, which is different than the hard fill material, to the exterior of the header assembly connector and allowing the seal material to cure.

18. The method of claim 17, wherein
the seal material defines a liquid state viscosity; and
the hard fill material defines a liquid state viscosity that is less than the seal material liquid state viscosity.

19. The method of claim 17, wherein
the seal material defines a cured state modulus; and
the hard fill material defines a cured state modulus that is greater than the seal material cured state modulus.

20. The method claimed in claim 17, wherein
the hard fill material comprises epoxy; and
the seal material comprises silicone adhesive.
